(19) United States
(12) Patent Application Publication
(10) Pub. No.: US 2007/0191682 A1
(43) Pub. Date: Aug. 16, 2007

(54) Optical probes for imaging narrow vessels or lumens

(76) Inventors: Jannick Rolland, Chuluota, FL (US); Huikai Xie, Gainesville, FL (US); Eric Gordon Johnson, Oviedo, FL (US); Olusegun Ilegbusi, Oviedo, FL (US); Kye-Sung Lee, Orlando, FL (US); Marco A. Costa, Vedra Beach, FL (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/417,599

(22) Filed: May 4, 2006

Related U.S. Application Data
(60) Provisional application No. 60/773,486, filed on Feb. 15, 2006.

(51) Int. Cl. A61B 1/06; A61B 1/00

(52) U.S. Cl. 600/173; 600/129; 600/130; 600/174; 600/182

(57) ABSTRACT

Disclosed are optical probes and methods for use of such probes. In one embodiment, an optical probe includes a housing that is sized and configured to be passed through a lumen having an inner diameter no greater than approximately 2 millimeters, and an internal optical system provided within the housing, the optical system being configured to capture images of a feature of interest associated with the lumen. In another embodiment, an optical probe includes a housing configured for passage through a narrow lumen, and an internal optical system provided within the housing that is configured to capture images of a feature of interest associated with the lumen, the optical system including axicon optics that form a focal line rather than a discrete focal point. In one embodiment, a method includes advancing an optical probe through the lumen to position the probe adjacent the feature of interest, and imaging the feature of interest across a depth of the feature of interest with invariance of resolution using an internal optical system of the probe.

### BACKGROUND

To date, it is believed that most myocardial infarctions result from the rupture of “vulnerable plaques,” that share certain common characteristics. These plaques typically comprise a lipid-rich core in the central portion of the thickened intima. This lesion contains an abundant amount of lipidladen macrophage foam cells derived from blood monocytes. The plaques have thin, friable fibrous caps and are therefore prone to rupture, triggered by inflammatory processes. Rupture of these plaques leads to an immediate clot formation with vessel obstruction and consecutive development of myocardial infarction.

Most vulnerable plaques are asymptomatic, obstructing less than about 70% of the vessel lumen. Stress analysis has demonstrated that when the intimal wall thickness is less than 70 microns (μm), susceptibility to rupture increases dramatically. However, current imaging technologies lack the capability to reliably identify these lesions.

In order to prevent subsequent cardiac events, there is need for a new imaging technology capable of identifying specific lesion types which are at risk of instability or progression, especially vulnerable plaques.

### SUMMARY

Disclosed are optical probes and methods for use of such probes. In one embodiment, an optical probe comprises a housing that is sized and configured to be passed through a lumen having an inner diameter no greater than approximately 2 millimeters, and an internal optical system provided within the housing, the optical system being configured to capture images of a feature of interest associated with the lumen.

In another embodiment, an optical probe comprises a housing configured for passage through a narrow lumen, and an internal optical system provided within the housing that is configured to capture images of a feature of interest associated with the lumen, the optical system including axicon optics that form a focal line rather than a discrete focal point.

In one embodiment, a method comprises advancing an optical probe through the lumen to position the probe adjacent the feature of interest, and imaging the feature of interest across a depth of the feature of interest with invariance of resolution using an internal optical system of the probe.

### BRIEF DESCRIPTION OF THE FIGURES

The components in the drawings are not necessarily to scale, emphasis instead being placed upon clearly illustrating the principles of the present disclosure. In the drawings, like reference numerals designate corresponding parts throughout the several views.

FIG. 1 is a perspective view of a first embodiment of an optical probe.

FIG. 2 is a side view of the optical probe of FIG. 1.

FIG. 3 is a perspective view of an optical system used in the optical probe shown in FIGS. 1 and 2.

FIG. 4 is a side view of an axicon lens used in the optical system of FIG. 3.

FIG. 5 is a modulation transfer function for the optical system shown in FIG. 3.

FIGS. 6A and 6B are illustrations of embodiments of use of the optical probe shown FIGS. 1 and 2 within a vessel or lumen.

FIG. 7 is a partial side view of a second embodiment of an optical probe.

FIG. 8 is a partial side view of a third embodiment of an optical probe.

FIG. 9 is an illustration of an alternative use of an optical probe.

FIG. 10 is a side view of a fourth embodiment of an optical probe.

FIG. 11 is a side view of a diffractive optical element, shown coupled to an axicon lens, that can be used in one or more of the optical probes.

### DETAILED DESCRIPTION

As described above, there is need for a new imaging technology capable of identifying specific lesions which are at risk of instability or progression, especially vulnerable plaques. Disclosed in the following is an optical probe that is well suited for use in identifying such lesions. Although the disclosed probe is suitable for such use, it is to be appreciated that the probe is capable of other uses, both biological and otherwise.

In the following, described are various embodiments of optical probes. Although particular embodiments of optical probes and the optical systems they comprise are described, those embodiments are mere example implementations of the disclosed probes and optical systems. Furthermore, the terminology used in this disclosure is selected for the purpose of describing the disclosed probes and optical systems and is not intended to limit the breadth of the disclosure.

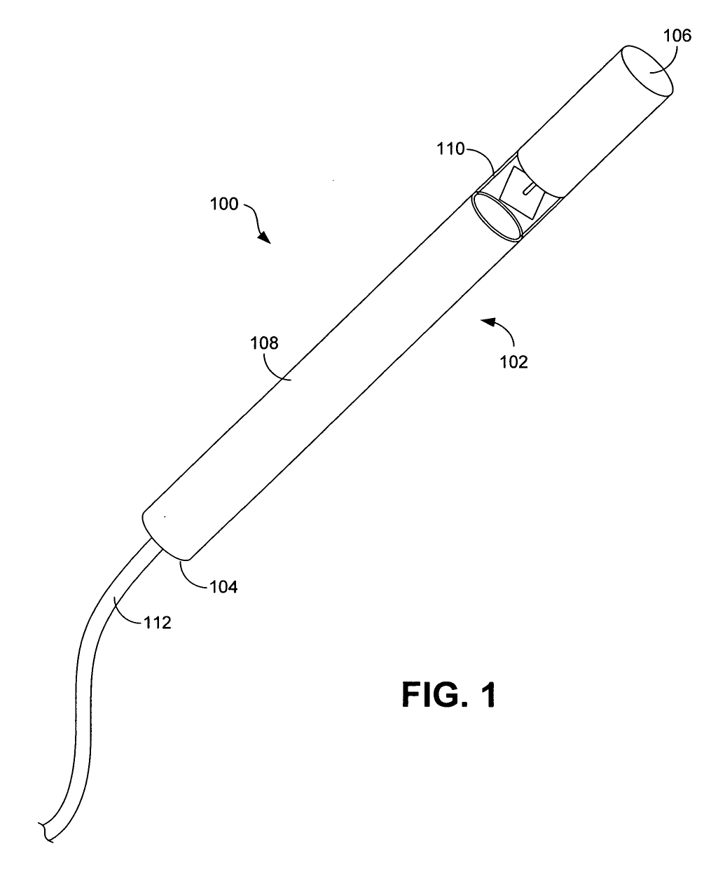

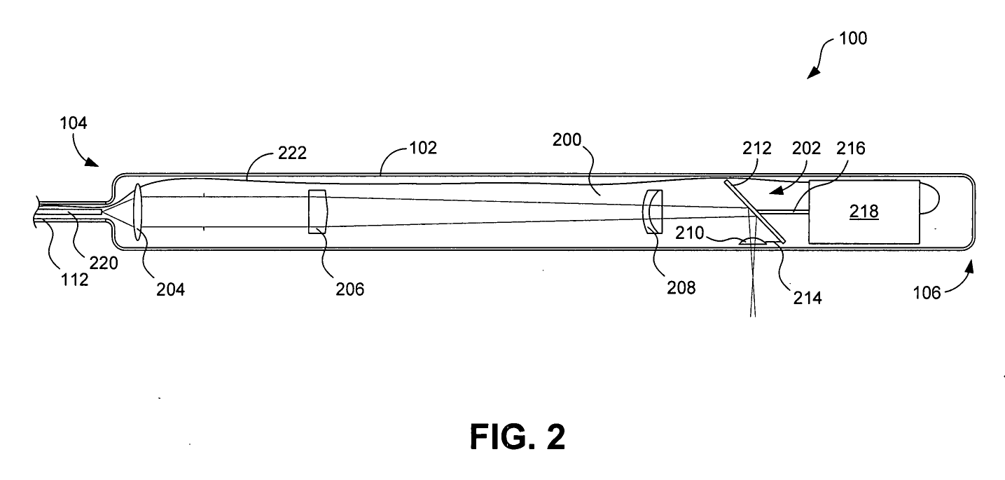

Beginning with FIG. 1, illustrated is an embodiment of an optical probe 100 that is suitable for use within narrow vessels or lumens, such as arteries, lung lobes, and other internal biological structures. As shown in FIG. 1, the probe 100 includes a generally cylindrical outer housing 102. The outer housing 102 is elongated and comprises a proximal end 104, a distal end 106, and an outer periphery 108 that extends between the two ends. In the illustrated embodiment, an imaging window 110 is provided along the outer periphery 108 adjacent the distal end 106 of the probe 100. Visible through the imaging window 110 in FIG. 1 are components of an internal optical system of the probe, that system being described in detail in relation to FIGS. 2-4 below. In the embodiment of FIG. 1, the imaging window 110 extends along the circumference of the outer housing 102 so as to permit 360° viewing using the internal optical system.

The optical probe 100 is dimensioned such that it may be used in narrow, for example small diameter, vessels or lumens. By way of example, the optical probe 100 has an outer diameter from approximately 1 millimeter (mm) to 2 mm, and a length of approximately 20 mm from its proximal end 104 to its distal end 106.

Extending from the proximal end 104 of the optical probe 100 is a flexible cord 112 that, as is described below, transmits light to and receives signals from the probe. The outer diameter of the cord 112 can be smaller than that of the probe 100, and the length of the cord can depend upon the particular application in which the probe is used. Generally speaking, however, the cord 112 is long enough to extend the probe 100 to a site to be imaged while the cord is still connected to a light source (not shown) that emits light through the cord to the probe.

The materials used to construct the optical probe 100 and its cord 112 can be varied to suit the particular application in which they are used. In biological applications, biocompatible materials are used to construct the probe 100 and cord 112. For example, the outer housing 102 of the probe 100 can be made of stainless steel or a biocompatible polymeric material. The imaging window 110 can be made of a suitable transparent material, such as glass, sapphire, or a clear, biocompatible polymeric material. In some embodiments, the material used to form the imaging window 110 can also be used to form a portion or the entirety of outer housing 102.

The cord 112 can comprise a lumen made of a resilient and/or flexible material, such as a biocompatible polymeric material. In some embodiments, the cord 112 can comprise a lumen composed of an inner metallic coil or braid, for example formed of stainless steel or nitinol, that is surrounded by an impermeable polymeric sheath. Such an embodiment provides additional column strength and kink resistance to the cord 112 to facilitate advancing the probe 100 to the imaging site. In addition, the outer housing 102 and/or the cord 112 can be coated with a lubricious coating to facilitate insertion and withdrawal of the probe to and from the imaging site.

FIG. 2 illustrates the interior 200 of the optical probe 100 and cord 112. As is shown in that figure, the probe 100 houses an internal optical system 202. In the embodiment of FIG. 2, the optical system comprises collimation optics including a collimating lens 204, axicon optics including an axicon lens 206, imaging optics including a first imaging lens 208 and a second imaging lens 210, and a mirror 212. Each of the collimating lens 204, axicon lens 206, and first imaging lens 208 are fixedly mounted within the housing appropriate mounting fixtures (not shown). Substantially any mounting fixtures that secure the lenses in place and that do not undesirably obstruct the transmission of light through the optical system 202 can be used. The second imaging lens 210 is fixedly mounted to the mirror 212 with a mounting arm 214 that extends from the mirror. The mirror 212 is, in turn, mounted to a shaft 216 of a micromotor 218 that is fixedly mounted adjacent the distal end 106 of the probe 100. As shown in FIG. 2, the mirror 212 is mounted to the shaft 216 such that the mirror reflects light rays transmitted by the first imaging lens 208 toward the distal end 106, and reflects light rays transmitted back from the second imaging lens toward the center of the probe 100.

Extending through the cord 112 is an optical waveguide 220, such as a single-mode optical fiber, and a power cord 222 that also extends through the optical probe 100 to the micromotor 218 to provide power to the micromotor. By way of example, the micromotor comprises a 1.9 mm Series 0206 micromotor produced by MicroMo Electronics, Inc.

With the above-described configuration, light from a high-intensity light source (not shown) is transmitted by the optical waveguide 220 to the collimating lens, to the first imaging lens 208, to the mirror 212, to the second imaging lens 210, and then out from the optical probe 100 to the imaging site (not shown). When the micromotor 218 is activated, it rotates the shaft 216 and, therefore, axially rotates the mirror 212 and the second imaging lens 210 about a longitudinal central axis of the probe 100 such that images can be captured substantially through 360° relative to that axis (i.e., the central axis extending from the proximal end 104 to the distal end 106).

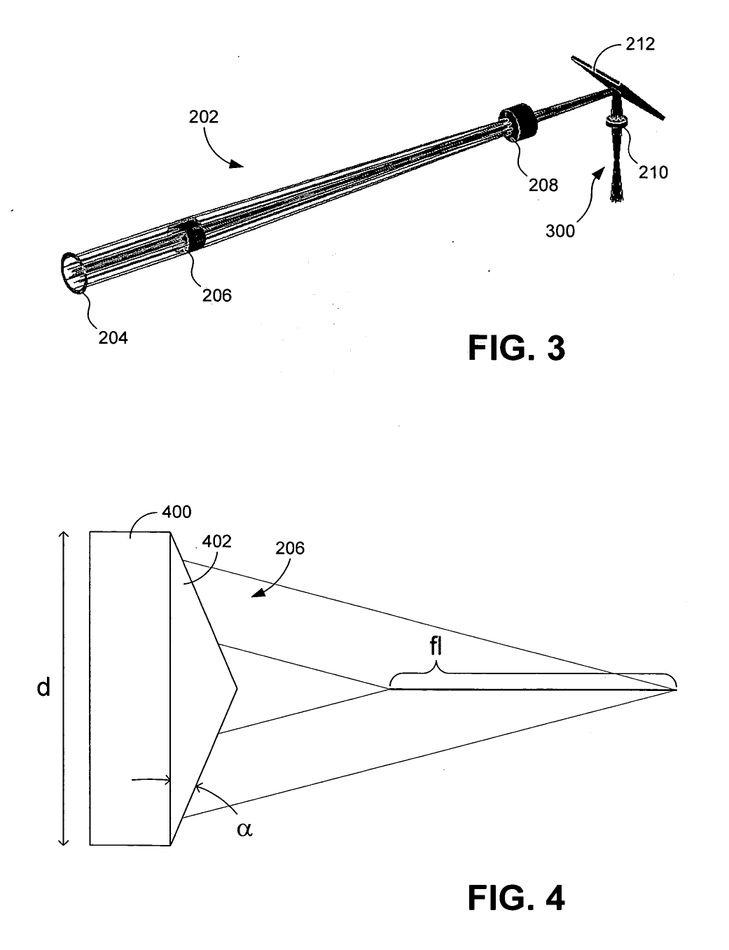

FIG. 3 depicts the transmission of light rays through the optical system 202 of the probe 100. As indicated in FIG. 3, the collimating lens 204 collimates light transmitted by the optical waveguide (220, FIG. 2) so as to deliver collimated light to the axicon lens 206. The axicon lens 206 focuses the light toward the first imaging lens 208, which, together with the second imaging lens 210, further focuses the light to create a displaced focal zone 300 in which features of interest may be imaged.

Important to the formation of the focal zone 300 is the axicon lens 206. The axicon lens 206 is illustrated in FIG. 4 (figure not to scale). As is depicted in FIG. 4, the axicon lens 206 comprises a generally cylindrical portion 400 and a generally conical portion 402 that is distal of the cylindrical portion. The conical portion 402 of the lens 206 tends to form a focal zone or focal line, fl, rather than a discrete focal point as is formed by typical spherical lenses. Due to the formation of a focal line rather than a focal point, a feature of interest can be imaged across substantially the entire focal line, instead of at just one point, with invariance of resolution. Because of that, dynamic focusing, and the various optical elements and mechanisms required to provide such dynamic focusing, are unnecessary. As a result, the size of the optical system 202 and the probe 100 in which it is used can be significantly reduced. Therefore, the use of the axicon lens 206 enables miniaturization of the probe 100 so as to enable its use in vessels or lumens having diameters of approximately 2 mm or less. In one embodiment, the axicon lens 206 has a diameter, d, of approximately 0.8 mm and an axicon angle, α, of approximately 3.16°.

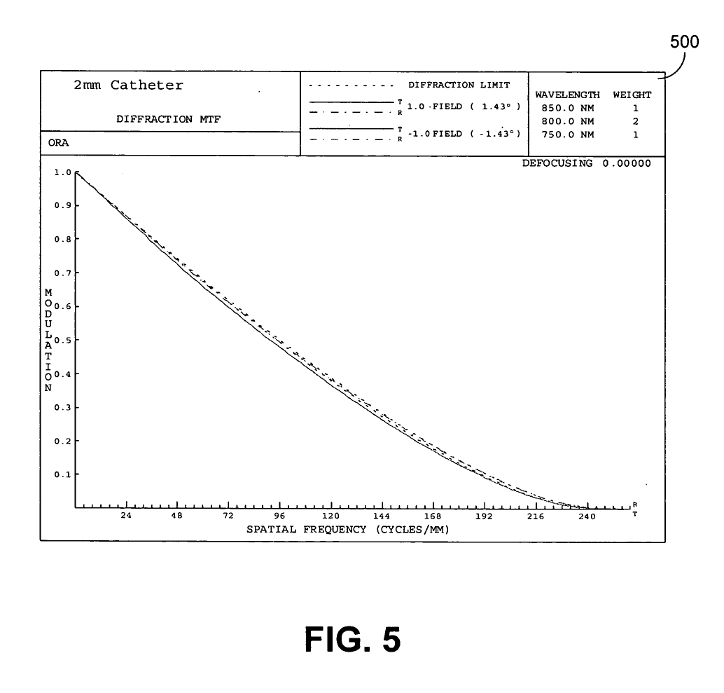

FIG. 5 provides a graph of the modulation transfer function (MTF) for the optical system 202. Plotted in the graph of FIG. 5 is the diffraction limit (dashed line) of the system 202 and frequency response curves of tangential (T) and sagittal (R) light rays. As is apparent from FIG. 5, the optical system 202 is well designed given that the MTF curves closely follow the diffraction limit curve.

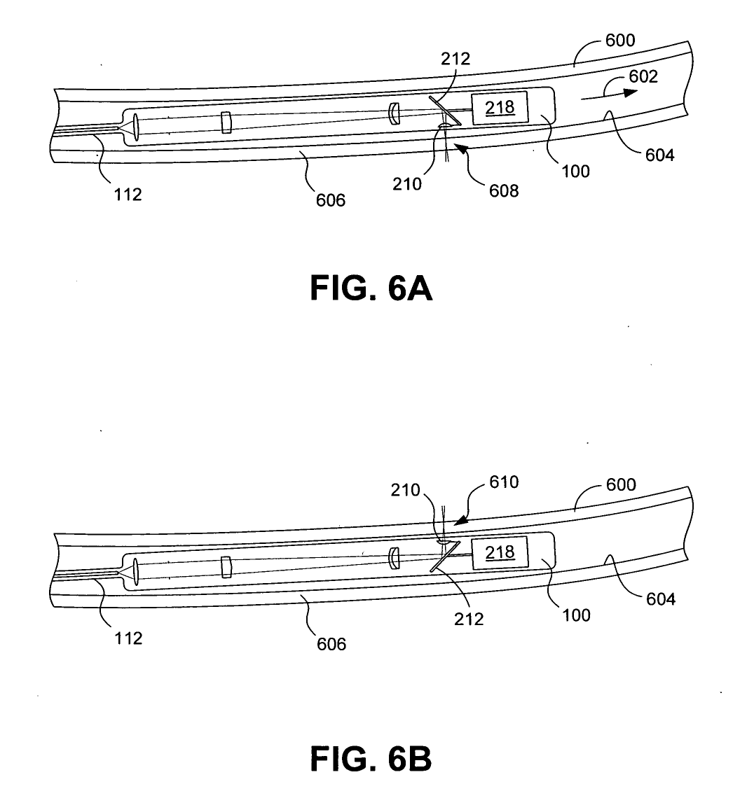

FIGS. 6A and 6B illustrate the optical probe 100 in use within a vessel or lumen 600. By way of example, the vessel or lumen 600 may comprise a human vessel, such as an artery or lung lobe. Although an artery and a lung lobe have been specifically identified, it is noted that the vessel or lumen can comprise an alternative vessel or lumen, whether it be biological or non-biological. Other biological vessels or lumens include veins as well as other canals or passageways formed within the body. In such biological applications, the optical probe 100 may be considered an optical catheter. Generally speaking, however, the optical probe 100 can be used to image features of interest in substantially any narrow vessel, lumen, or passageway.

Referring first to FIG. 6A, the optical probe 100 is shown positioned within the vessel or lumen 600. For biological applications, the probe 100 can have been positioned by introducing the probe into the vessel or lumen 600 using a needle or trocar (not shown). Once so introduced, the probe 100 can be placed into position along the vessel or lumen 600 by advancing the probe using the cord 112, for example in the direction indicated by arrow 602. Optionally, appropriate external visualization techniques, such as x-ray imaging, can be used to guide in the practitioner positioning the probe 100 at the desired imaging site.

Once the optical probe 100 is positioned as desired, the inner surface 604 and/or interior 606 of the wall that forms the vessel or lumen 600 can be imaged using the probe. In FIG. 6A, the interior 606 of a bottom portion 608 of the vessel or lumen is imaged with the probe 100. As is apparent from that figure, the focal zone of the optical system 202 coincides with the wall interior 606 such that a given depth of the wall can be imaged without the need to adjust focus. By way of example, a resolution of approximately 5 μm can be achieved across a focal line or depth up to approximately 2 mm. For instance, in one embodiment, a resolution of 4.8 μm can be achieved for a focal line or depth of 1.5 mm.

Turning to FIG. 6B, the mirror 212 and second imaging lens 210 have been rotated 180° relative to their positions illustrated in FIG. 6A such that a second portion 610 of the vessel or lumen wall is imaged. Again, the wall interior 606 is imaged across a depth instead of at a discrete point such that dynamic focusing is unnecessary. Although only two portions 608 and 610 of the wall 606 have been illustrated as being imaged using the optical probe 100, it is to be understood that the entire circumference of the vessel or lumen 600 can be imaged in the same manner due to the 360° rotation capability of the mirror 212 and the second imaging lens 210. Therefore, in some embodiments, images may be continually captured as the mirror 212 and second imaging lens 210 are continuously rotated or “swept” by the micromotor 218.

Various imaging technologies may be used to form images of the features of interest. In some embodiments, optical coherence tomography (OCT) or optical coherence microscopy (OCM) can be desirable. OCT and OCM are non-contact, light-based imaging modalities that gather two-dimensional, cross-sectional imaging information from target tissues or materials. In medical and biological applications, OCT or OCM can be used to study tissues in vivo without having to excise the tissue from the patient or host organism. Since light can penetrate tissues to varying degrees, depending on the tissue type, it is possible to visualize internal microstructures without physically penetrating the outer, protective layers. OCT and OCM, like ultrasound, produces images from backscattered “echoes,” but uses infrared (IR) or near infrared (NIR) light, rather than sound, which is reflected from internal microstructures within biological tissues, specimens, or materials. While standard electronic techniques are adequate for processing ultrasonic echoes that travel at the speed of sound, interferometric techniques are used to extract the reflected optical signals from the infrared light used in OCT or OCM. The output, measured by an interferometer, is computer processed to produce high-resolution, real-time, cross-sectional, or three-dimensional images of the tissue. Thereby, OCT or OCM can provide in situ images of tissues at near histologic resolution.

For a detailed discussion of OCT as used in biological applications, refer to “Optical Coherence Tomography (OCT),” by Ulrich Gerckens et al., Herz, 2003, which is hereby incorporated by reference into the present disclosure. In embodiments in which OCT is used, IR or NIR light emitted from a high-intensity light source, such as a super-luminescent diode or a laser, can be transmitted through the optical system 202. By way of example, a Gaussian beam having a central wavelength of approximately 800 nanometers (nm) to 1500 nm can be used. Notably, video rates can be achieved in cases in which Fourier-domain OCT is performed.

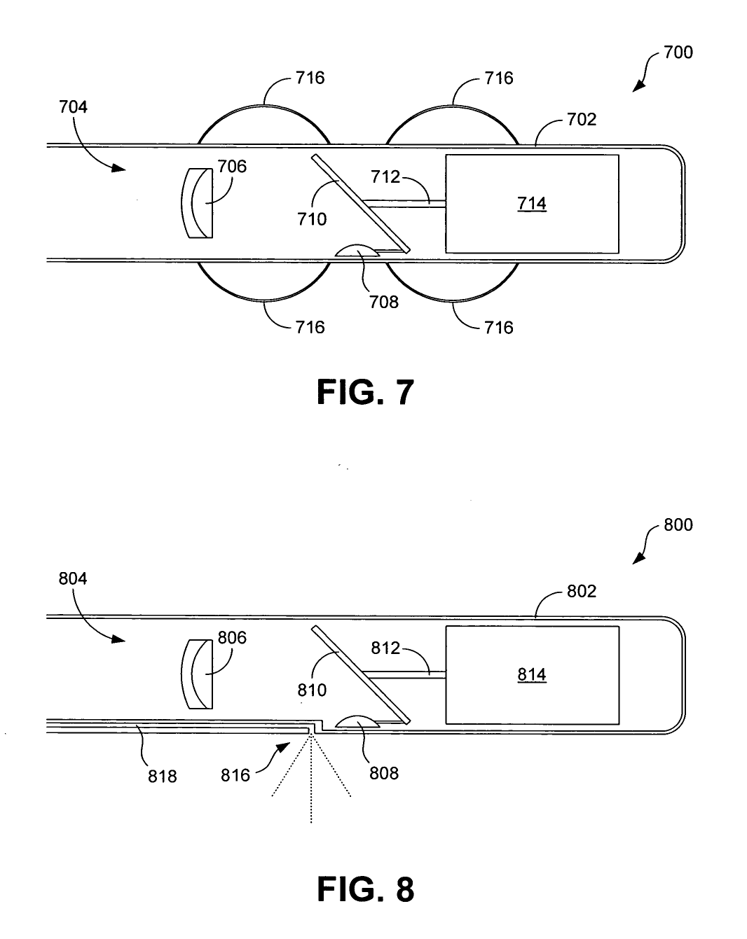

Turning to FIG. 7, illustrated is an alternative optical probe 700. The optical probe 700 is similar to the optical probe 100 and, therefore, includes an outer housing 702 and an internal optical system 704 that includes imaging optics having a first and second imaging lenses 706 and 708, and a mirror 710. The mirror 710 and second imaging lens 706 are driven by a shaft 712 connected to a micromotor 714. In addition, however, the optical probe 700 includes balloons 716, shown in an inflated state. The balloons 716 can be selectively inflated with a suitable fluid, such as air or saline, and are fed by supply lumens (not shown). When the balloons 716 are inflated, they can block the flow of fluid, such as blood, through the vessel or lumen in which the probe 700 is disposed to facilitate imaging of the interior surface or internal structure of the vessel or lumen wall. Accordingly, the probe 700 can be placed in a first position along the length of the vessel or lumen, the balloons 716 can be inflated, images can be captured through 360°, the balloons can be deflated, and the probe can be moved to a second position along the length of the vessel or lumen to repeat the imaging process. Notably, the balloons 716 surround the circumference of the outer housing 702 such that the flow of fluid can be completely blocked. Although two balloons 702 are illustrated in FIG. 7, a single balloon can be used, either proximal or distal of the second imaging lens 708, as desired.

FIG. 8 illustrates a further alternative optical probe 800. The optical probe 800 is also similar to the optical probe 100 and, therefore, includes an outer housing 802 and an internal optical system 804 that includes imaging optics having a first and second imaging lenses 806 and 808, and a mirror 810. The mirror 810 and second imaging lens 806 are driven by a shaft 812 connected to a micromotor 814. In addition, however, the probe 800 includes a fluid port 816 that is configured to eject clear fluid, such as saline, adjacent the imaging site to dilute the fluid, such as blood, present at the imaging site. The port 816 can be fed via an internal channel or lumen 818 provided within the outer housing 802 and the probe's cord (not shown). Although a single port 816 is illustrated proximal of the second imaging lens 808 in FIG. 8, a port can additionally or alternatively be provided distal of the second imaging lens 808, if desired. In addition or in alternative, the probe 800 can include multiple ports or a continuous port that is/are provided around the periphery of the probe.

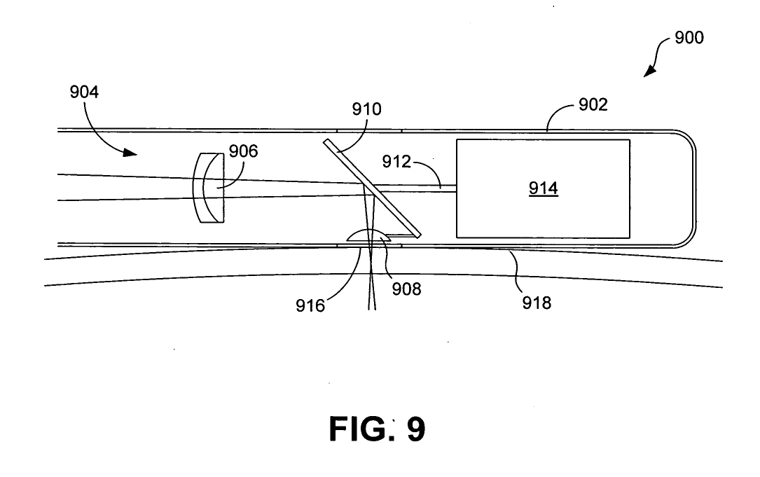

FIG. 9 illustrates an alternative use of an optical probe 900. The optical probe 900 is also similar to the optical probe 100 and, therefore, includes an outer housing 902 and an internal optical system 904 that includes imaging optics having a first and second imaging lenses 906 and 908, and a mirror 910. The mirror 910 and second imaging lens 906 are driven by a shaft 912 connected to a micromotor 914. In the illustrated use, the outer housing 902, and therefore the imaging window 916, are placed substantially in contact with a wall 918 of a vessel or lumen to be imaged. In such a case, balloons or irrigation means as described above in relation to FIGS. 7 and 8, respectively, may not be necessary. Once images have been capture of the wall 918, the probe 900 can be repositioned against another portion of the wall and the image capture process repeated.

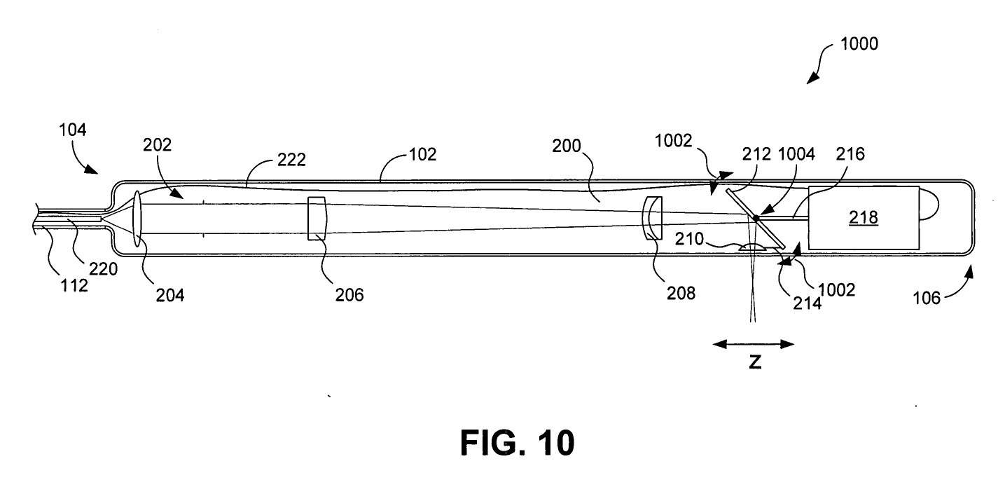

Turning to FIG. 10, illustrated is a further alternative optical probe 1000. The optical probe 1000 is similar to the optical probe 100 and, therefore, includes several of the components that are described in relation to FIG. 2, those components having similar construction and function in the embodiment of FIG. 10. Unlike the optical probe 100, however, the optical probe 1000 is capable of scanning a feature of interest along a direction that is substantially parallel to the central axis of the probe, i.e., the “z” direction indicated in FIG. 10. Such scanning is possible through pivoting of the mirror 212 about an axis 1004 that is substantially perpendicular to the central axis of the probe, as indicated by directional arrows 1002. As is apparent from FIG. 10, pivoting of the mirror 212 in the clockwise direction will enable leftward scanning (in the orientation of the figure), while pivoting of the mirror in the counterclockwise direction will enable rightward scanning (in the orientation of the figure). That scanning, coupled with rotation of the mirror 212 in the manner described above, enables three-dimensional imaging of the vessel or lumen in which the probe 1000 is disposed.

Pivoting of the mirror 212 can be achieved using various different pivoting mechanisms. By way of example, the pivoting mechanism can include microelectromechanical systems (MEMS) components (not shown) that pivot the mirror 212 within a frame (not shown) to which the mirror is pivotally mounted. Optionally, the second imaging lens 210 can be fixedly mounted to that frame such that the mirror 212 and lens can be pivoted together in unison.

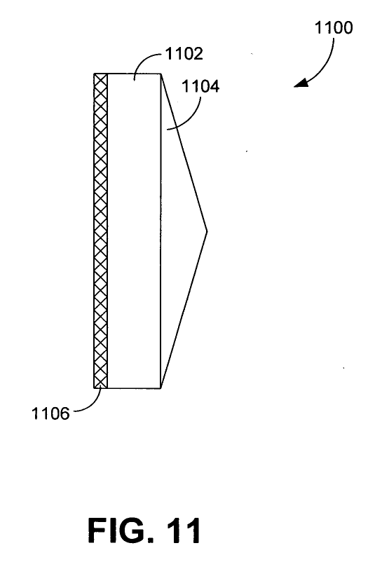

Turning to FIG. 11, illustrated is a diffractive optical element 1106 that corrects chromatic aberration in the optical system in which it is used. As indicated in FIG. 11, the diffractive optical element 1106 can be coupled to or formed on an axicon lens 1100 of the optical system, the lens comprising a cylindrical portion 1102 and a conical portion 1104. Although the diffractive optical element 1106 is shown being coupled to or formed on an axicon lens, the diffractive optical element could be provided elsewhere in the optical system. For example, with reference to FIG. 2, the diffractive optical element 1106 could, alternatively, be coupled to or formed on a side of the collimating lens 204 that faces the axicon lens 206.

As noted above, while particular embodiments have been described in this disclosure, alternative embodiments are possible. For example, alternative embodiments may combine features of the discrete embodiments described in the foregoing. Therefore, an optical probe may comprise, for instance, balloons and irrigation means. In addition, although imaging of vessel or lumen “walls” has been described, the principles disclosed herein can be applied to other features, such as growths or deposits formed on or within such walls. All alternative embodiments are intended to be covered by the present disclosure.

1. An optical probe comprising:
a housing that is sized and configured to be passed through a lumen having an inner diameter no greater than approximately 2 millimeters; and
an internal optical system provided within the housing, the optical system being configured to capture images of a feature of interest associated with the lumen.

2. The probe of claim 1, wherein the housing is generally cylindrical.

3. The probe of claim 2, wherein the housing is approximately 1 millimeter to 2 millimeters in diameter.

4. The probe of claim 1, wherein the housing includes an imaging window through which images can be captured by the internal optical system.

5. The probe of claim 1, wherein the internal optical system is configured to capture images around a circumference of the housing.

6. The probe of claim 1, wherein at least a portion of the internal optical system is axially rotatable about a central axis of the probe to enable imaging through 360° relative to the central axis.

7. The probe of claim 6, further comprising a micromotor provided within the housing that rotates the at least a portion of the internal optical system.

8. The probe of claim 6, wherein the at least a portion of the optical system further is pivotable about an axis substantially perpendicular to the central axis of the probe to enable scanning of the lumen in a direction substantially parallel to the central axis of the probe.

9. The probe of claim 1, wherein the internal optical system comprises an axicon lens that forms a focal line rather than a discrete focal point.

10. The probe of claim 1, further comprising a flexible cord that extends from the housing and surrounds an optical waveguide that delivers light from an external light source to the internal optical system.

11. The probe of claim 1, wherein the housing further comprises a selectively-inflatable balloon configured to block the flow of fluid through the lumen.

12. The probe of claim 1, wherein the housing further comprises a fluid port configured to eject fluid to dilute other fluid within the lumen adjacent the feature of interest.

13. An optical probe comprising:
a housing configured for passage through a narrow lumen; and
an internal optical system provided within the housing that is configured to capture images of a feature of interest associated with the lumen, the optical system including axicon optics that form a focal line rather than a discrete focal point.

14. The probe of claim 13, wherein the housing is approximately 1 millimeter to 2 millimeters in diameter.

15. The probe of claim 13, wherein the housing includes an imaging window through which images can be captured by the internal optical system.

16. The probe of claim 13, wherein the internal optical system further comprises collimating optics that collimate light before it reaches the axicon optics.

17. The probe of claim 13, wherein the internal optical system further comprises imaging optics that receive light transmitted by the axicon optics.

18. The probe of claim 17, wherein the imaging optics comprise a first imaging lens and a second imaging lens.

19. The probe of claim 13, wherein the internal optical system further comprises a mirror that reflects light transmitted by the optical system toward the feature of interest.

20. The probe of claim 18, wherein the mirror is axially rotatable relative to a central axis of the probe such that images of the lumen can be captured around a circumference of the probe.

21. The probe of claim 20, further comprising a micromotor provided within the housing that axially rotates the mirror.

22. The probe of claim 20, wherein the mirror further is pivotable about an axis substantially perpendicular to the central axis of the probe such that images of the lumen can be captured along a direction substantially parallel to the central axis of the probe.

23. The probe of claim 13, further comprising a flexible cord that extends from the housing and surrounds an optical waveguide that delivers light from an external light source to the internal optical system.

24. The probe of claim 13, wherein the housing further comprises a selectively-inflatable balloon configured to block the flow of fluid through the lumen.

25. The probe of claim 13, wherein the housing further comprises a fluid port configured to eject fluid to dilute other fluid within the lumen adjacent the feature of interest.

26. An optical probe comprising:
a housing having an outer diameter no greater than approximately 2 millimeters; and
an internal optical system provided within the housing that is configured to capture images of a feature of interest associated with the lumen, the optical system including
collimating optics that collimate light emitted by a light source,
axicon optics that focus the light along a focal line as opposed to a discrete point,
imaging optics that displace the focal line created by the axicon optics, and
a mirror that reflects light transmitted by the optical system out toward the feature of interest.

27. The probe of claim 26, wherein the mirror is axially rotatable about a central axis of the probe such that the direction at which light is emitted from the housing can be adjusted to enable image capture around a circumference of the housing.

28. The probe of claim 27, wherein the mirror further is pivotable about an axis substantially perpendicular to the central axis of the probe such that images of the lumen can be captured along a direction substantially parallel to the central axis of the probe.

29. The probe of claim 26, wherein the imaging optics comprise a first imaging lens and a second imaging lens.

30. The probe of claim 29, wherein the second imaging lens is mounted to the mirror so as to be axially rotatable with the mirror.

31. The probe of claim 27, further comprising a micromotor provided within the housing that rotates the mirror.

32. The probe of claim 26, further comprising a flexible cord that extends from the housing and surrounds an optical waveguide that delivers light from an external light source to the internal optical system.

33. The probe of claim 26, wherein the housing further comprises a selectively-inflatable balloon configured to block the flow of fluid through the lumen.

34. The probe of claim 26, wherein the housing further comprises a fluid port configured to eject fluid to dilute other fluid within the lumen adjacent the feature of interest.

35. A method for imaging a feature of interest of a lumen, comprising:
advancing an optical probe through the lumen to position the probe adjacent the feature of interest; and
imaging the feature of interest across a depth of the feature of interest with invariance of resolution using an internal optical system of the probe.

36. The method of claim 35, wherein advancing an optical probe comprises advancing the optical probe through a human vessel.

37. The method of claim 35, wherein advancing an optical probe comprises advancing the optical probe through an artery or a lung lobe.

38. The method of claim 35, wherein advancing an optical probe comprises advancing an optical probe through a lumen having an inner diameter no greater than approximately 2 millimeters.

39. The method of claim 35, wherein advancing an optical probe comprises advancing an optical probe having a diameter of approximately 1.5 millimeters to 2 millimeters.

40. The method of claim 35, wherein imaging the feature of interest comprises imaging the feature of interest at a resolution of approximately 5 microns across a focal line of approximately 1.5 millimeters to 2 millimeters.

41. The method of claim 35, wherein imaging the feature of interest using an optical system comprises imaging the feature of interest using an optical system comprising axicon optics that create a focal line rather than a discrete focal point.

42. The method of claim 35, wherein imaging the feature of interest comprises imaging the feature of interest using optical coherence tomography (OCT).

43. The method of claim 35, further comprising circumferentially imaging the lumen through rotation of a portion of an internal optical system of the optical probe.

44. The method of claim 43, further comprising linearly imaging the lumen through pivoting of the portion of the optical system.
